(19) United States
(12) Patent Application Publication
(10) Pub. No.: US 2021/0054324 A1
(43) Pub. Date: Feb. 25, 2021

(54) 3D SPATIALLY PATTERNED LYMPH NODE ON A MICROFLUIDIC DEVICE

(71) Applicant: University of Virginia Patent Foundation, Charlottesville, VA (US)

(72) Inventors: Rebecca Rose Pompano, Charlottesville, VA (US); Jennifer Ortiz, Charlottesville, VA (US)

(21) Appl. No.: 17/045,459

(22) PCT Filed: Apr. 5, 2019
(86) PCT No.: PCT/US2019/026170

§ 371 Date: Oct. 5, 2020

Related U.S. Application Data
(60) Provisional application No. 62/652,980, filed on Apr. 5, 2018.

(51) Int. Cl. C12M 3/06; C12M 1/12; C12M 1/00; C12N 5/00; G01N 33/50

(52) U.S. Cl. CPC C12M 23/16; C12M 25/14; C12M 29/00; C12N 2513/00; G01N 33/5082; G01N 33/5047; C12N 5/0062

(57) ABSTRACT

Provided are microfluidic chip-based models of immune system tissues. In some embodiments, the models include a microfluidic housing having a cell culture chamber and one or more channels in communication with the cell culture chamber and a cell culture residing in the cell culture chamber, wherein the cell culture includes one or more cells of the immune system. Also provided are systems that include one or more microfluidic chip-based models of immune system tissues, methods for patterning cells in culture on microfluidic chips, and methods for modeling immune responses of subjects using the same.

### CROSS-REFERENCE TO RELATED APPLICATION

This application claims the benefit of U.S. Provisional Patent Application Ser. No. 62/652,980, filed Apr. 5, 2018, the disclosure of which is incorporated herein by reference in its entirety.

### TECHNICAL FIELD

The presently disclosed subject matter relates to microfluidic chip-based models of tissues of the immune system, including models of lymph nodes and the spleen. The presently disclosed subject matter also relates to systems comprising the disclosed microfluidic chip-based models, methods for making the same, and methods for using the same to model immune responses in subjects including mammalian subjects.

### BACKGROUND

Events in the lymph node (LN) are central to human health. They determine how well mammals fight infections and respond to vaccines, whether a nascent tumor is recognized and destroyed, and whether tissues are protected without autoimmunity. The cell types and molecules comprising the node are intricately organized, with dynamically defined zones for T cells, B cells, and antigen presenting cells (Qi et al. (2014) Annu Rev Cell Dev Biol 30:141-167; Willard-Mack (2006) Toxicol Pathol 34:409-424). These are subject to lymphatic and interstitial flows that modulate local signaling and chemotaxis (Swartz et al. (2008) Semin Immunol 20:147-156; Tomei et al. (2009) J Immunol 183:4273-4283; Lund et al. (2016) J Clin Invest 126:3389-3402; Thomas et al. (2016) Annu Rev Biomed Eng 18:207-233). T cells enter the node from blood vessels and must migrate through the tissue, searching for their target, called an antigen (Germain et al. (2008) Immunol Rev 221:163-181). This migration occurs appears to follow a random walk (Cahalan & Parker (2008) Annu Rev Immunol 26:585-626), but actually follows a labyrinthine underlying network of stromal cells (Kaldjian et al. (2001) Int Immunol 13:1243-1253; Bajénoff et al. (2006) Immunity 25:989-1001). These basic organizational elements of the lymph node were essential to the design of the artificial lymph node chip.

Both antibody production and killer T cell generation are initiated in the lymph node. Events therein determine how well mammals fight infections and respond to vaccines, whether a nascent tumor is recognized and destroyed, and whether the individual's own tissues remain safe from autoimmunity (Cochran et al. (2006) Nat Rev Immunol 6:659-670; Benaglio et al. (2015) BioMed Res Int 2015:e420251; Groom (2015) Immunol Cell Biol 93:330-306). The lymph node is emerging as an attractive target for immunomodulation and immunotherapy (Swartz et al. (2012) Sci Transl Med 4:148rv9; Andorko et al. (2014) AAPS J 2014:1-16; Andorko et al. (2016) Cell Mol Bioeng 9:418-432), but it remains difficult to study the function and dynamics of this organ outside of animal models.

The potential to model the human body on a microchip offers tantalizing hope of predictive drug testing and unprecedented control for mechanistic experiments. However, the growing collection of organ-on-chip systems still lacks the lymph node (LN), the small and highly organized organ that initiates adaptive immune responses. Without a LN chip, the nascent human-on-chip cannot replicate fundamental B cell- or T cell-mediated immunity. In addition, few in vitro systems enable direct functional investigation of the complex molecular and cellular events that control immunity. An experimentally tractable, biomimetic chip model of the dynamics and organization of this organ is needed both for mechanistic studies and to test new therapies.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides microfluidic chip-based models of tissues of the immune system. In some embodiments, the microfluidic chip-based models comprise a microfluidic housing comprising a cell culture chamber and one or more channels in communication with the cell culture chamber; and a cell culture residing in the cell culture chamber, the cell culture comprising one or more cells of the immune system. In some embodiments, the cell culture comprises a three-dimensional (3D) cell culture. In some embodiments, the one or more cells of the immune system are patterned within the cell culture. In some embodiments, the one or more cells of the immune system comprise and/or are selected from the group consisting of healthy and/or naive (resting) cells of the immune system; primary immune cells, optionally wherein the primary immune cells are B cells, T cells, dendritic cells, macrophages, and/or neutrophils; genetically modified cells, optionally cells obtained from a genetically modified animal and/or cells modified by a CRISPR/Cas9 method; a diseased cell; an activated cell, optionally a cell collected from a donor that has been treated with a drug or other therapy, or a donor that is a disease model, or a cell that was treated in vitro with a drug or therapy; or any combination thereof.

In some embodiments of the presently disclosed subject matter, the one or more cells of the immune system are derived from a murine or human source, the one or more cells of the immune system are derived from stem cells, optionally induced pluripotent stem cells, and/or the one or more cells of the immune system are derived from a diseased donor.

In some embodiments of the presently disclosed subject matter, the one or more cells of the immune system are patterned in one or more zones, optionally wherein one or more channels pass through the one or more zones and/or wherein one or more channels surround the one or more zones. In some embodiments, a first zone comprising a first type of the one or more cells of the immune system is surrounded by a second zone of a second type of the one or more cells of the immune system, optionally wherein the first type of the one or more cells of the immune system is B cells and the second type of the one or more cells of the immune system is T cells. In some embodiments, the first zone and the second zone are surrounded by a third zone, optionally wherein the third zone is a sinus. In some embodiments, the cell culture comprises one or more non-immune cell types, optionally stromal cells and/or endothelial cells and/or neural cells (optionally neurons).

In some embodiments of the presently disclosed subject matter, the cell culture comprises a scaffold, optionally wherein the scaffold is formed from a scaffold-forming composition that comprises a reagent that induces and/or facilitates and/or enhances pattern formation, further optionally a photo-reactive crosslinking group and/or a photoinitiator; optionally wherein the scaffold comprises a protein-based biomaterial scaffold, further optionally a gelatin methacrylate, a gelatin norbornene, and/or a collagen; and/or optionally wherein the scaffold comprises a non-protein material, further optionally polyethylene glycol. In some embodiments, the scaffold can optionally contain additional structural elements such as fibers and particles at the nano and/or microscale, which in some embodiments can provide support for, facilitate, or otherwise enhance migration of cells within the cell culture. In some embodiments, the cell culture comprises a mixture of scaffolds, optionally wherein the scaffolds are dispersed homogeneously through the cell culture or are patterned in one or more different zones of the cell culture. In some embodiments, the scaffold-forming composition can also comprise additional components besides those required to form the scaffold itself, such as but not limited to one or more chemokines, cell-binding moieties, receptor ligands, inhibitors, enzymes, active agents such as but not limited to drugs, and combinations thereof. For example, in some embodiments heparin is present in the scaffold. Heparin serves to bind chemokines, which are secreted by the cells. Alternatively or in addition, chemokines can also be added deliberately to the scaffold, for example to add additional functionalities.

In some embodiments of the presently disclosed subject matter, the microfluidic housing comprises a material selected from the group consisting of a silicone rubber (in some embodiments polydimethyl siloxane), glass, a thermoplastic polymer, a three-dimensional (3D) printed resin, a biomaterial (in some embodiments a PEG-based material or a protein-based material), and a combination thereof.

In some embodiments of the presently disclosed subject matter, the microfluidic housing comprises one or more surfaces having a surface chemistry modification. By way of example and not limitation, a methacrylated silane can be added to the surface to enable gelatin methacrylate (GelMA) to covalently bind to the interior surface of the chip. In some embodiments, a thiolated silane can be employed for the same purpose when gelatin norbornene (GelNB) is employed. In some embodiments, an interior surface of the chip can be coat with a protein or a mixture of proteins, such as but not limited to coating the interior surface with serum albumin and/or pretreating the interior surface with culture media that contains serum.

In some embodiments, the microfluidic housing comprises at least two channels in communication with the cell culture chamber, wherein at least one of the channels is configured to deliver input to the cell culture chamber and wherein at least one of the channels is configured to receive output from the cell culture chamber.

In some embodiments, the microfluidic housing is assembled either with irreversible bonding or with a reversible method such as but not limited to clamping. In some embodiments, a clamped system is advantageous as it enables one to retrieve the gel from the housing for further analysis such as, but not limited to by flow cytometry or high-magnification imaging.

In some embodiments, the microfluidic housing comprises one or more additional chambers in communication with the cell culture chamber, where one or more of the channels is configured to deliver input to the cell culture chamber from the one or more additional chambers and/or one or more of the channels is configured to deliver output from the cell culture chamber to the one or more additional chambers. In some embodiments, the one or more channels in communication with the cell culture chamber comprise one or more channels configured to deliver input to one or more zones in the cell culture, optionally wherein a first zone comprising a first type of the one or more cells of the immune system is surrounded by a second zone of a second type of the one or more cells of the immune system, further optionally wherein the first type of the one or more cells of the immune system is B cells and the second type of the one or more cells of the immune system is T cells. In some embodiments, the first zone and the second zone are surrounded by a third zone, optionally wherein the third zone is a sinus.

In some embodiments, the microfluidic housing comprises one or more additional layers comprising one or more additional channels and/or one or more additional chambers, wherein one or more additional layers are disposed above and/or below a first layer comprising the cell culture chamber and the one or more channels in communication with the cell culture chamber. In some embodiments, the one or more additional channels and/or one or more additional chambers are in communication with the cell culture chamber and the one or more channels in communication with the cell culture chamber of the first layer, optionally wherein the communication is achieved via a valve, a port, and/or a semi-permeable membrane between the layers.

In some embodiments of the presently disclosed subject matter, the models represent a length scale model of a mammalian lymph node, optionally wherein the lymph node is selected from the group consisting of a skin-draining lymph node, a cervical lymph node, a mesenteric lymph node, an iliac lymph node, a mediastinal lymph node, a popliteal lymph node, a tonsil, a Peyer's patch, and a tertiary lymphoid structure that forms in sites of chronic inflammation. In some embodiments, the mammalian lymph node that is modeled is a human lymph node.

In some embodiments of the presently disclosed subject matter, the models provide a model of mammalian spleen tissue, optionally human spleen tissue.

In some embodiments, the presently disclosed subject matter further comprises one or more pumps externally disposed relative to the microfluidic housing, the one more pumps communicating with the one or more channels for moving reagents into the one more channels.

In some embodiments, the presently disclosed subject matter also provides systems comprising one or more models as disclosed herein and one or more other tissue-on-chip devices. In some embodiments, a model as disclosed herein is in communication with the one or more other tissue-on-chip devices, optionally wherein the system is used to model multi-tissue immunity and/or multi-tissue responses to a drug. In some embodiments, the one or more other tissue-on-chip devices are selected from the group consisting of a liver device, a kidney device, a lung device, a brain device, and a tumor device. In some embodiments, a system of the presently disclosed subject matter comprises one or more models as disclosed herein in communication with the one or more other tissue-on-chip devices, and further comprises one or more other devices, such as but not limited to a computer and/or a processor that in some embodiments can be used in controlling the system, a reagent introduction device such as but not limited to a pump assembly. In some embodiments, the computer and/or the processor is operatively connected reagent introduction device and can provide control of fluidic flow from the reagent introduction device to the model. In some embodiments, the reagent introduction device comprises a pump assembly that itself comprises one or more peristaltic pumps and is in fluidic communication with the one or more channels for moving reagents into the one or more channels of the model. For providing two or more reagents to the model, the pump assembly can comprise at least two or more pumps. The model can in some embodiments be mounted on a microscope stage. In some embodiments, the microscope comprises a microscope stage that can be controllably actuated in the X-Y and/or the X-Y-Z space to align the model with the reagent introduction device and/or any other component of the system as might be desired.

In some embodiments, the presently disclosed subject matter also provides methods for patterning cells in cultures on microfluidic chips. In some embodiments, the methods comprise providing a microfluidic housing comprising a cell culture chamber and one or more channels in communication with the cell culture chamber; and patterning cells in the cell culture chamber, optionally wherein the cells comprise one or more cells of the immune system, to provide a culture in the cell culture chamber. In some embodiments, the culture is a three-dimensional (3D) culture. In some embodiments, the patterning comprises directing a composition comprising one or more cells of the immune system to the cell culture chamber through the one or more channels; wherein the composition comprises a reagent that induces pattern formation, optionally wherein the composition comprises one or more non-immune cell types, optionally stromal cells and/or endothelial cells and/or neural cells (e.g., neurons). In some embodiments, the patterning comprises directing light through a photomask covering a portion of the microfluidic housing, providing one or more architectural features in the cell culture chamber, modifying surface chemistry of the microfluidic housing, and any combination thereof. In some embodiments, the light directed through the photomask has a wavelength ranging from about 300 nanometers (nm) to about 800 nm, optionally about 405 nm. In some embodiments, the photomask is selected from the group consisting of a printed transparency mask, a chrome mask, a digital mask, and another standard mask used for photolithography. In some embodiments, the patterning comprises altering the surface chemistry of the microfluidic housing and wherein the reagent that induces pattern formation comprises a moiety that reacts with the altered surface chemistry.

In some embodiments, the presently disclosed subject matter also provides methods for modeling immune responses of a subject. In some embodiments, the methods comprise providing a model of a tissue of the immune system or a system as disclosed herein; delivering a stimulus to the model or to the model that is part of the system; and evaluating a response to the stimulus. In some embodiments, delivering the stimulus comprises delivering a vaccine to the model or system; and evaluating the response comprises evaluating one or more cell motility, gene expression, protein secretion, small molecule secretion, production of reactive oxygen species, and metabolic activity. In some embodiments, delivering the stimulus comprises delivering an established or new therapy or drug to the model or system. In some embodiments, delivering the stimulus comprises testing spatial organization of the model or system, optionally wherein delivering the stimulus comprises varying a location and/or an inclusion of a particular cell type between a first model or system and a subsequent model or system, and/or varying a rate and/or distribution of fluidic flow through the model or system.

In some embodiments, the model is patterned using healthy and/or naive (resting) cells to model a healthy tissue of the immune system. In some embodiments, the model is patterned with cells from a diseased donor to model that disease. In some embodiments, the model is patterned with genetically modified cells to model a disease and/or to test a hypothesis about a role of a modified gene or pathway.

Thus, it is an object of the presently disclosed subject matter to provide microfluidic chip-based models of tissues of the immune system.

An object of the presently disclosed subject matter having been stated herein above, and which is achieved in whole or in part by the presently disclosed subject matter, other objects will become evident as the description proceeds when taken in connection with the accompanying Figures as best described herein below.

### BRIEF DESCRIPTION OF THE FIGURES

FIGS. 1A-1C depict a model of the intricate structure of the lymph node (LN) in a lymph-node-on-a-chip device of the presently disclosed subject matter. FIG. 1A depicts the intricate structure of a LN, which facilitates rare interactions (Qi et al. (2014) Annu Rev Cell Dev Biol 30(1):141-167). With respect to either mice or humans, the LN comprises, at a basic level, discrete B cells zones surrounded by T cells zones surrounded by a lymphatic sinus. Blood vessels also traverse the LN, which also includes a medulla. FIG. 1B is a fluorescence micrograph of a lymph node section that was stained with fluorescent primary antibodies to reveal the structure of B cell and T cell zones (Groff et al. (2019) J Immunol Methods 464:119-125). FIG. 1C is a schematic drawing of a micro-patterned 3D cell culture model within a microfluidic device of the presently disclosed subject matter. B cell zones are shown as three darker ovals, with the T cell zone (lighter gray) surrounding the B cells. The lymphatic sinus (white area) surrounds the B cell and T cell zones. Also depicts are afferent lymphatics (three, with arrows pointing towards the B and T cell zones) as well as am efferent lymphatic/outlet (with an arrow pointing away from the B and T cell zones). In about the middle of the micro-patterned 3D cell culture model is a white oval surrounded by a gray border, which mimics the central circulatory vessels of the LN.

FIGS. 2A and 2B are a schematic of prototype version of Lymph Node-on-Chip microfluidic housing (FIG. 2A) and an image of an assembled prototype LN on chip device, fabricated on a glass layer with a polydimethylsiloxane (PDMS) layer (FIG. 2B).

FIGS. 3A-3D depict patterning on a chip. FIG. 3A is a brightfield image of an exemplary lymph node chamber near two afferent channels. 200-μm microposts divide the interior region from the “sinus”. FIG. 3B is a fluorescent image of device filled with 8% NHS-rhodamine-labelled GelMA (red in color; light gray in FIG. 3B). Microposts exclude the gel precursor from the sinus region (black area inside of the larger broken white circle). The photomask with two circular follicles can be seen faintly below (outlined in smaller dotted white lines inside the dotted box). FIG. 3C is a photograph of a patterned 8% GelMA labeled with NHS Rhodamine (red; dark gray circles) and NHS Fluorescein (green; remaining white area). Scale bar is 1 mm. FIG. 3D is a patterned “B cell follicle” (area enclosed by dashed line) with NHS Rhodamine labeled cells. Surrounding paracortex region with CFSE labeled cells, 25×10⁶ cells/ml. Scalebar 500 μm.

FIGS. 4A-4D are schematic drawings depicting controlling fluidic flow and spatial patterning to test mechanisms of the immune response using models in accordance with the presently disclosed subject matter. In FIG. 4A, an exemplary chip that is based on a healthy node is depicted. B cell zones shown as dark gray circles, T cell zone is the slightly lighter gray surrounding the B cell zones in green, with the sinus in white surrounding the T cell zone. On-chip vaccination with OVA plus LPS was predicted to produce cytokines (gray haze) and slow T cell motility (hypothetical paths that cells could have followed are depicted as lines in FIGS. 4A and 4C. FIGS. 4B-4D are exemplary modified designs of chip models in accordance with the presently disclosed subject matter for testing the effects of different flow rates, directions, and spatial structures and/or patterning, respectively.

FIG. 5 is a schematic of an exemplary on-chip photolithography method for patterning a lymph-node-on-a-chip device (LN-chip) of the presently disclosed subject matter. Briefly, the device is filled with buffer with other inlets sealed to prevent backflow (Panel i). The syringe containing the precursor solution with the first population of cells (e.g., B cells and/or precursors thereof) is plugged into the remaining inlet, and its pumped into the device, displacing the buffer (Panel ii). A mask made of a transparency material with the design of interest is placed on the back of the glass layer and the precursor solution is exposed to 405 nm wavelength light (Panel iii). The device is filled with buffer, removing any un-crosslinked material, resulting in the device returning to the state depicted in Panel i, but with one or more zones of cells patterned on an inner surface of the device (Panel iv). Optionally, the steps depicted in Panels ii-iv are repeated one or more times, each time with a new precursor solution containing a cell population of interest (e.g., T cells and/or precursors thereof) to pattern the cell population(s) of interest on the surface of the device (Panel v) In each subsequent iteration of the steps depicted in Panels ii-iv, a different transparency mask can be employed on the back of the glass layer, thereby exposing only those areas desired for patterning while simultaneously covering features already patterned. Buffer can then be pumped into the device to remove any excess material, resulting in a patterned LN-chip of the presently disclosed subject matter with, for example, one or more B cell zones surrounded by a T cell zone, both of which are encircled by a sinus.

FIGS. 6A-6D depict UV-photopatterning of an LN-chip of the presently disclosed subject matter chip directly in a microfluidic device. FIG. 6A is a bright-field image of the empty culture chamber near an incoming “lymphatic” channel (inlet). FIG. 6B is a fluorescent image of a device filled with 10% NHS-rhodamine-labelled GelMA (red in a color figure; light gray area in FIG. 6B). A photomask with a circular follicle can aligned beneath the chip (white circular area). FIG. 6C depicts producing a patterned GelMA follicle (outlined by dotted black line and labeled B), after which the chip was rinsed, refilled with 10% NHS-Fluoroscein-labelled GelMA, and exposed through a second mask to define T cell zone (T) and sinus. In a separate experiment, GelMA containing red- and green-labelled splenocytes were patterned similarly. FIG. 6D is a fluorescence micrograph of a patterned LN-chip in which the cells within the B cell zone (B) denoted with the broken white line are virtually all B cells and the cells outside the B cell zone (T) are virtually all T cells. Scale bar is 400 μm for FIGS. 6A-6C and 550 μm for FIG. 6D.

FIG. 6E is a bar graph of that quantification of cells intended for the B cell zone that were found inside of the T cell zone at 4° C. (left bar of each pair) or at 25° C. (right bar of each pair) as a measure of non-specific cell adhesion. Error bars represent standard deviation from the mean (n=2).

FIGS. 7A-7C are a series of graphs presenting the results of experiments of photopatterned in gelatin methacrylate (GelMA) as a culture matrix or scaffold on the LN-chip. FIG. 7A is a graph of the storage modulus of GelMA (8% lower trace; 10% upper trace) crosslinked with 0.1% lithium phenyl-2,4,6-trimethylbenzoylphosphinate (LAP) as a photoinitiator at 10 mW/cm² and increasing time. Exposure of 10% GelMA at 2700 mJ/cm² provided the desired stiffness. Dashed lines in the Figure show the errors (standard deviation from the mean). FIG. 7B is a bar graph of murine splenocyte viability 1 hour after off-chip gelation at 405 nm, measured by a Calcein AM/Propidium Iodide assay, was within 20% of fresh cells. Light dosage held constant at 2700 mJ/cm² by varying intensity and time inversely. Error bars represent standard deviation from the mean. FIG. 7C is a bar graph of cell-laden GelMA patterned on-chip as in FIG. 6 and rested 1 hour. Error bars represent standard deviation from the mean. *p<0.05.

FIGS. 8A and 8B depict fluidic flow rates through an exemplary LN-chip. A 2D simulation of fluidic flow through a 10-mm LN-chip, conducted with finite element method in COMSOL Multiphysics. The tissue was modeled as a porous matrix and the lymphatics and sinus as open channels. Incoming flow velocity through the afferent lymphatics was set to 5 μm/second (Roadmap Epigenomics Consortium, Kundaje et al. (2015) Integrative analysis of 111 reference human epigenomes. Nature 518:317-330). FIG. 8A is a plot showing predicted uniform velocity (μm/s) over the surface of the culture chamber. FIG. 8B is a velocity profile along the dotted line in FIG. 8A, which was located in the B cell region. The predicted interstitial flow velocity was ˜0.5 μm/sec in the hydrogel and 4.5 μm/s in the sinus, which closely matched physiological values (Farh et al. (2015) Genetic and epigenetic fine mapping of causal autoimmune disease variants. Nature 518:337-343).

FIGS. 9A and 9B depict patterning lymphatic channels and stroma on an exemplary LN-chip of the presently disclosed subject matter. FIG. 9A depicts patterning of stromal cells and simple vessel through the exemplary LN-chip. Arrows show directions of fluidic flow through the exemplary LN-chip. FIG. 9B is a photograph of an open channel micropatterned in cell-laden GelMA on-chip of mixed splenocytes at 10×10⁶ cells/mL. Inset shows Calcein/PI stain showing individual cells. Scale bar is 50 FDC: follicular dendritic cells. FRC: fibroblastic reticular cells. In vivo, FRCs are located only in the T cell zone, and FDCs are located only in the B cell zones. Thus, FIG. 9A depicts the B cell zones as comprising FDCs and the T cell zones as comprising FRCs.

FIG. 10 is a schematic exploded view of an organ model wherein the model is provided in three layers.

FIG. 11 is a schematic view of an exemplary organ model system provided in accordance with embodiments disclosed herein.

### DETAILED DESCRIPTION

All references listed in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter. Following long-standing patent law convention, the terms “a”, “an”, and “the” refer to “one or more” when used in this application, including in the claims. For example, the phrase “a cell” refers to one or more cells, including a plurality of the cells. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition and are encompassed within the nature of the phrase “consisting essentially of”.

With respect to the terms “comprising”, “consisting of”, and “consisting essentially of”, where one of these three terms is used herein, the presently disclosed and claimed subject matter can include the use of either of the other two terms. For example, in some embodiments, the presently disclosed subject matter relates to compositions comprising antibodies. It would be understood by one of ordinary skill in the art after review of the instant disclosure that the presently disclosed subject matter thus encompasses compositions that consist essentially of the antibodies of the presently disclosed subject matter, as well as compositions that consist of the antibodies of the presently disclosed subject matter.

The term “chamber” as used herein refers to a site where two or more substances are exposed to one another. The term also refers to a portion along a surface that is capable of maintaining a substance therein or there along. The “chamber” can take on a physical structure such as a hole, a well, a cavity, or an indentation, and have any cross-sectional shape along its length, width, or depth, such as rectangular, circular, or triangular. The term “between” when used in the context of moving between “a first position” and a “second position” can refer to movement only from a first position to a second position, movement only from a second position to a first position, or movement from a first position to a second position and from the second position to the first position. In some embodiments, a chamber is a culture chamber. In some embodiments, the culture chamber has an external wall that is about 100 μm in height.

The terms “react” and “reaction” refer to a physical, chemical, biochemical, and/or biological transformations that involves at least one substance, e.g., reactant, reagent, phase, carrier fluid, or plug-fluid and that generally involves (in the case of chemical, biochemical, and biological transformations) the breaking or formation of one or more bonds such as covalent, noncovalent, van der Waals, hydrogen, or ionic bonds. The term includes typical photochemical and electrochemical reactions, typical chemical reactions such as synthetic reactions, neutralization reactions, decomposition reactions, displacement reactions, reduction-oxidation reactions, precipitation, crystallization, combustion reactions, and polymerization reactions, as well as covalent and non-covalent binding, phase change, color change, phase formation, dissolution, light emission, changes of light absorption or emissive properties, temperature change or heat absorption or emission, conformational change, and folding or unfolding of a macromolecule such as a protein.

The term “substance” as used herein refers to any chemical, compound, mixture, solution, emulsion, dispersion, suspension, molecule, ion, dimer, macromolecule such as a polymer or protein, biomolecule, precipitate, crystal, chemical moiety or group, particle, nanoparticle, reagent, reaction product, solvent, or fluid, and any one of which can exist in the solid, liquid, or gaseous state, and which is typically the subject of an analysis.

The term “exposed” as used herein is a form of communication between two or more elements. These elements can in some embodiments include a substance, a chamber, a duct, a passage, a channel, a sinus, a lumen, or any combination thereof. In some embodiments, “exposed” can mean that two or more substances are in fluidic communication with each other, or alternatively, in some embodiments it can mean that two or more substances react with one another.

The term “fluidic communication,” as used herein, refers to any duct, channel, tube, pipe, or pathway through which a substance, such as a liquid, gas, or solid can pass substantially unrestricted when the pathway is open. When the pathway is closed, the substance is substantially restricted from passing through. In embodiments where a substrate is present, a substance can pass from one chamber to another through the substrate when the device is in the closed position, if the chambers are spatially positioned to allow diffusion via the substrate versus passage via a pathway. Typically, limited diffusion of a substance through the material of a plate, base, and/or a substrate, which in some embodiments can or in some embodiments cannot occur depending on the compositions of the substance and materials, does not constitute fluidic communication.

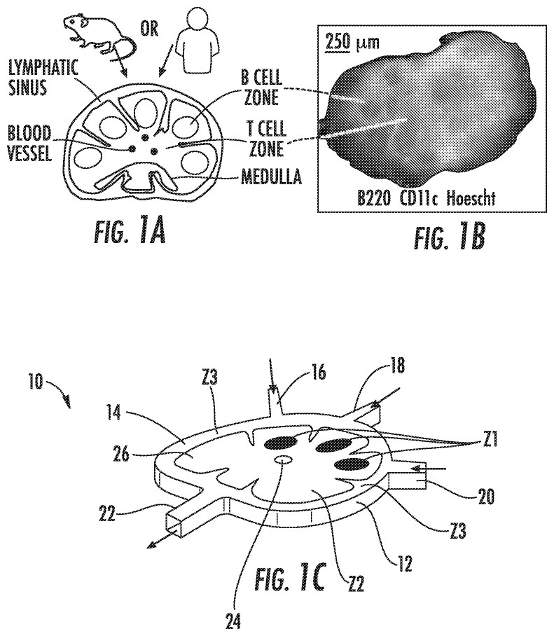

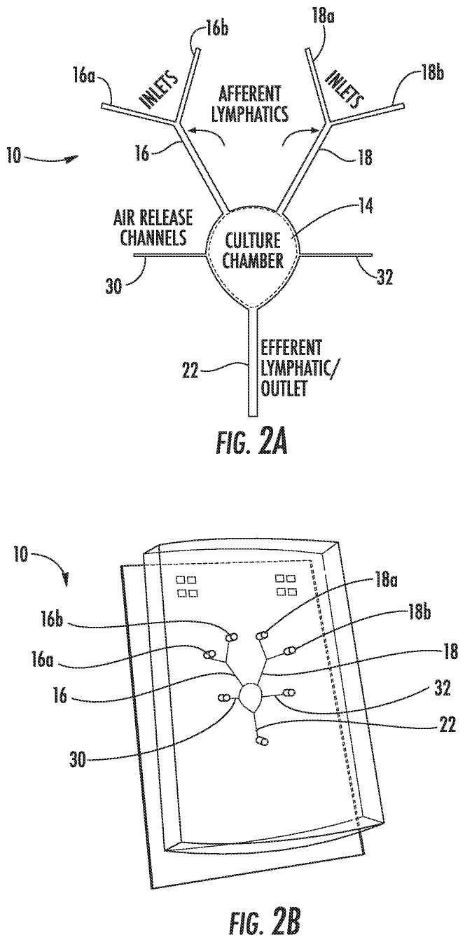

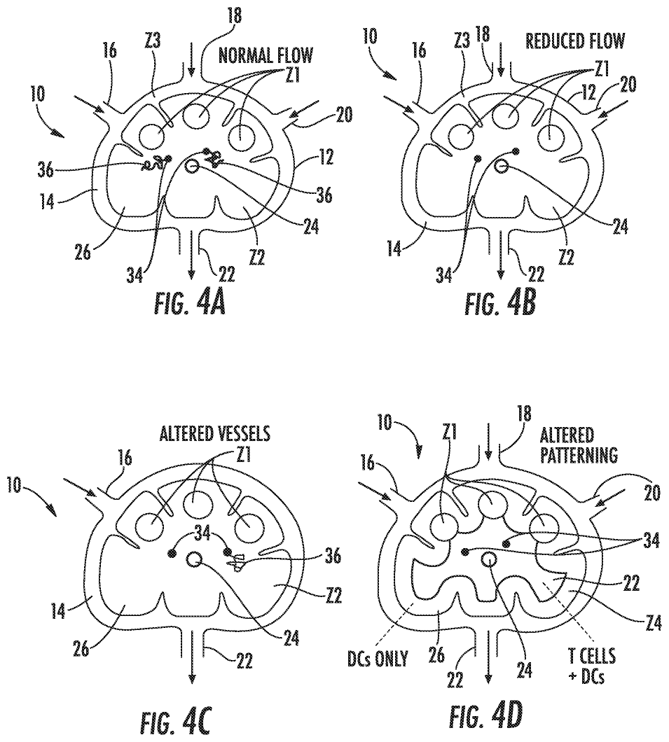

Referring now to the drawings, wherein like reference numbers refer to like parts throughout, and referring in particular to FIG. 1C through FIG. 3C, FIGS. 4A through 5, FIG. 8A, FIG. 9A, and FIG. 9B, a model of a tissue of an immune system in accordance with the presently disclosed subject matter is generally referred to as 10. Model 10 comprises a microfluidic housing 12, which comprises a cell culture chamber 14 and one or more channels in communication with cell culture chamber 14. With particular reference to FIG. 1C, 4A, and 5, the one or more channels comprise first channel 16, second channel 18, third channel 20, fourth channel 22, and fifth channel 24. In some embodiments, fifth channel 24 is designed to mimic the function of a blood vessel in an immune tissue in vivo. In some embodiments, the channels can be further divided into inlets. In this regard reference is made to FIG. 2A wherein channels first channel 16 and second channel 18 each further comprise inlet channels 16A and 16B, and 18A and 18B, respectively. Additionally, in some embodiments, such as the embodiment referred to in FIGS. 2A and 2B, the one or more channels can further comprise air release channels 30 and 32.

Continuing with reference to FIG. 1C, and FIGS. 4A through 4D, model 10 further comprises cell culture 26. Cell culture 26 comprises zones Z1 and Z2, wherein patterned cells are provided. For example, zone Z1 can comprise B cells and/or precursors thereof, and zone Z2 can comprise T cells and/or precursors thereof. Zone Z3 is a channel that defines a sinus that surrounds the cells of zone Z1 and zone Z2. Channel 24 passes through zones Z1 and Z2. Referring particularly to FIG. 4D a fourth zone, zone Z4, is provided which can include another type of cell, such as a dendritic cell. Thus, in some embodiments, such as shown in FIG. 4D, altered patterning is provided.

Continuing with particular reference to FIGS. 4A through 4D, different fluidic flow patterns are shown. In this regard it is noted that the arrows in each of the Figures represent the flow of fluid. Thus channels 16, 18, and 20 in FIGS. 1C and FIGS. 4A and 4B show delivery of input to cell culture chamber 14 while channel 22 shows the receipt of output from chamber 14 and out of model 10. FIG. 4A suggests a normal flow for the lymphatic fluid flowing from model 10 while FIG. 4B shows a reduced flow with use of smaller or reduced sized arrows. Thus, in some embodiments it is possible to deliver a stimulus to the model by varying the rates and/or distribution of fluidic flow through the model or system. With particular reference to FIG. 4C an alternate vessel pattern is shown through the inclusion of only first channel 16 and fourth channel 22 and fifth channel 24, which again show input via channel 16 and output via channel 22. In any embodiment or in any of the Figures, channel 24 can be used for input or output. In any embodiment or in any of the Figures, channel 24 mimics the function of a blood vessel in model 10.

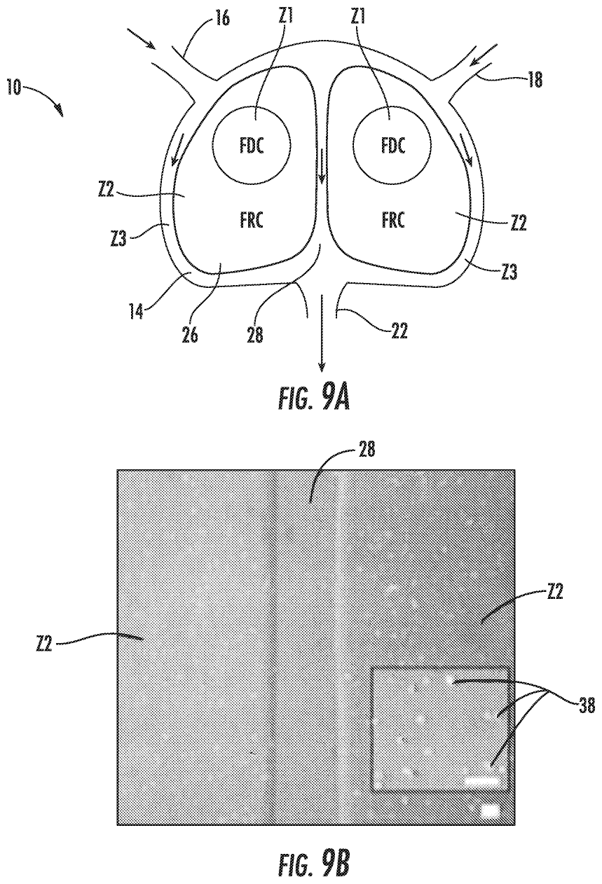

Continuing with particular reference to FIGS. 9A and 9B, sixth channel 28 can be included, which intersects and subdivides zone Z1 and zone Z2. In some embodiments, sixth channel 28 is designed to mimic a lymphatic vessel in an in vivo tissue of the immune system. Although FIGS. 9A and 9B show an exemplary model 10 with a single sixth channel 28, it is understood that model 10 can be designed to have additional sixth channels 28 that further intersect and subdivide zone Z1, zone Z2, or both zone Z1 and zone Z2, as desired. Furthermore, model 10 can thus be designed to include one or more of channels 16, 18, and 20 in conjunction with one or more of additional sixth channels 28 in such a way as to deliver a plurality of different types of fluids (optionally containing discrete or overlapping substances of interest) to various regions of model 10. The inset of FIG. 9B also shows individual cells 38 (e.g., splenocytes).

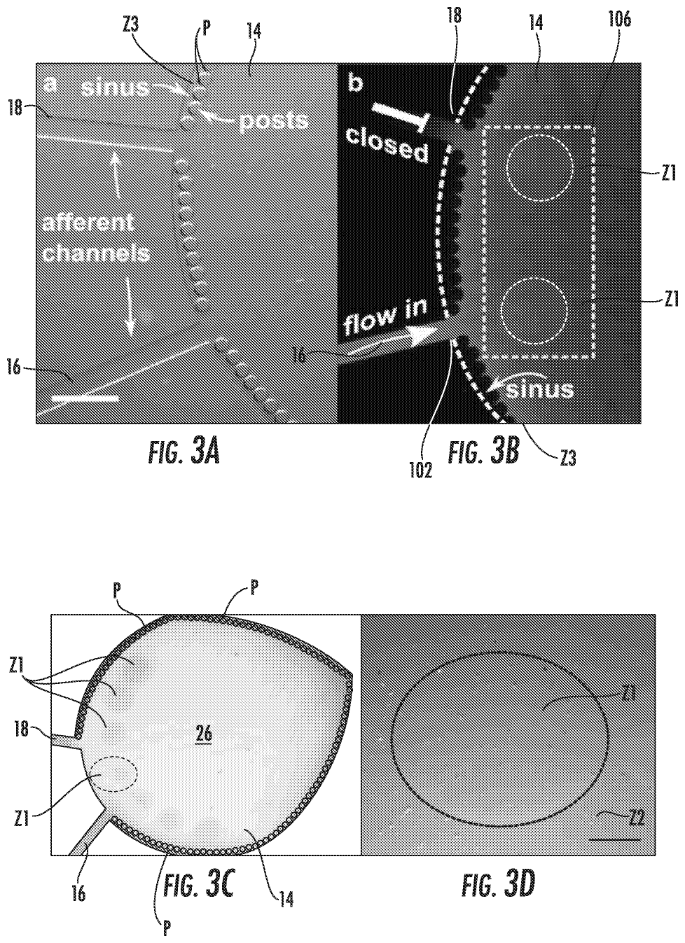

With particular reference to FIGS. 3A through 3D, input channels 16 and 18 are shown in communication with chamber 14. Reference is additionally made in FIGS. 3A through 3D to posts P, which are formed in chamber 14 and further so serve to define sinus zone Z3, which facilitates air release. Preparation of model 10 is initially described in FIG. 3B, which shows the flow in of cell precursor composition 102 via channel 16 to enter chamber 14 where zones Z1 are formed via mask 106 and exposure to a light source (not shown in FIG. 3B). FIG. 3C shows the formation of B cell zones Z1 to define a cell culture 26 in chamber 14. Referring again to FIG. 3B, the closure of channel 18 is also depicted in a representative embodiment for the preparation of model 10 in that the cell precursor composition 102 can only be allowed to flow through channel 16 if desired to facilitate the formation of zones Z1 comprising the cells.

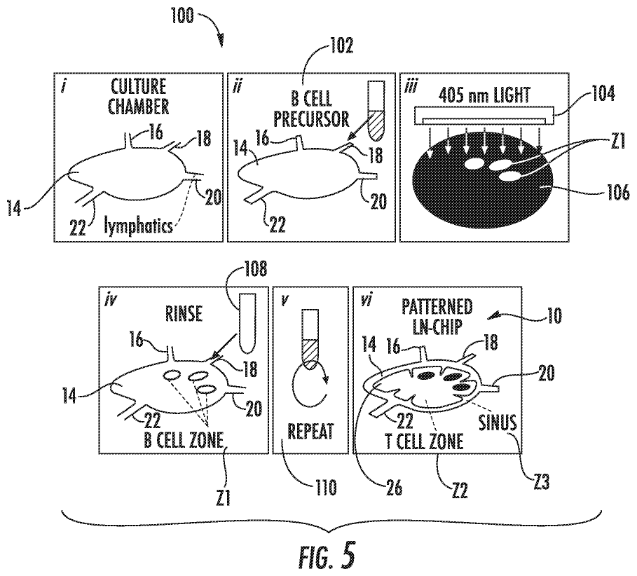

Continuing with reference to a process for preparing model 10 reference is now made to FIG. 5 wherein a representative process for the preparation of model 10 is referred to generally as process 100. In the first step of process 100 (Panel i), culture chamber 14 is provided which includes inlet channels 16, 18, and 20 and outlet channel 22. Proceeding with process 100 (Panel ii), B cell precursor composition 102 is flowed into chamber 14 via channels 16, 18, and 20 in this step the outflow of B cell precursor composition 102 through channel 22 is temporarily blocked. Referring to process step 100 (Panel iii), light source 104 is employed after mask defining zones Z1 is placed over chamber 14. B cell precursor composition 102 comprises a reagent that induces pattern formation optionally a photo reactive crosslinking group and a photo initiator. Thus, in some embodiments the cell precursor composition 102 serves to provide various reagents that generate a scaffold for cell culture 26 that is ultimately formed. Upon application of light from light source 104, cells in B cell precursor composition 102 are formed in zones Z1 as defined by mask 106.

Continuing with reference to process 100 (Panel iv) in FIG. 5, a rinse 108 is introduced to culture chamber 14 to drive out the cell precursor 102 that was not exposed to light from light source 104 and thus patterned in culture chamber 14. Channel 22 is opened to allow rinse 108 to flow into chamber 14 through channels 16, 18, and 20 and out through channel 22. In process 100 (Panel v), the steps are repeated with a solution of a T cell precursor composition 110 which also includes a photo reactive crosslinking group and photo initiator to provide a scaffold for culture 26. In this case, although not shown in FIG. 5, a mask that is effectively the reverse of the mask used in mask 106 is employed wherein zone one is covered but zone two remains open to be exposed to light so that T cells are provided in zone Z2. Here as well, T cell precursor composition 110 flows into chamber 14 via channels 16, 18, and 20 with channel 22 plugged. After light treatment channel 22 is opened and a buffer, again not shown in FIG. 5, is flowed through chamber 14 to remove T cell precursor 110 and ultimately to provide cell culture 26 and model 10. Sinus Z3 is also provided to facilitate air release.

Referring again to FIGS. 4A through 4D model 10 can further comprise a scaffold formed from pattern formation-inducing reagents as described elsewhere herein. In this case, the culture and/or scaffold can further comprise nanofibrous materials. In the FIGS. 4A through 4D, a mixture of scaffolds are provided and are patterned in zone Z2. However, it is possible that the scaffolds can be dispersed homogeneously throughout cell culture 26. Further patterning can be accomplished via the same techniques as disclosed herein with respect to patterning of cells.

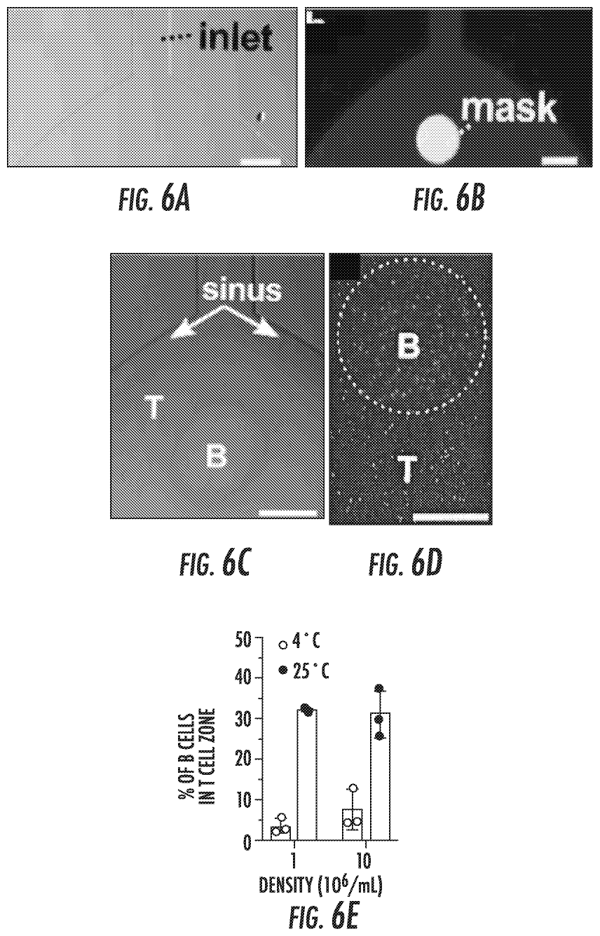

In some embodiments, zone Z2 is a T cell zone (see also FIG. 6C; T) comprising a plurality of T cells 34. Zone Z2 is in some embodiments designed to replicate the basic cellular functions of the T cell zone of a mammalian lymph node, including but not limited to replicating motility of T cell 34 as an indicator of the state and health of the tissue. At rest, T cell 34 would be expected to follow a modified random walk pattern with ˜10 μm/min average velocity (depicted as walk pattern 36 in FIGS. 4A and 4C).

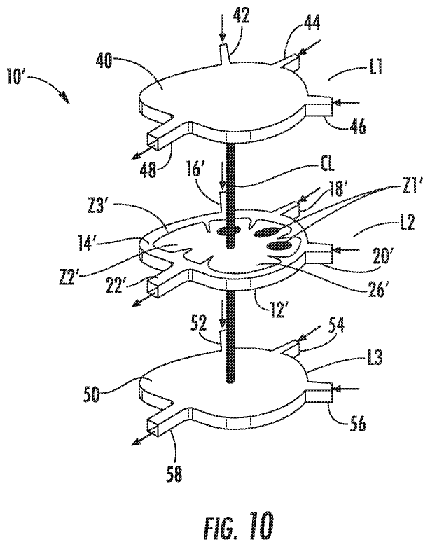

Referring now to FIG. 10, an exploded view of an embodiment of model 10′ comprising multiple layers is illustrated. In this embodiment, one or more additional layers of channels and chambers is provided above and/or beneath the cell culture chamber 14′, with channels passing downward and upward to emulate blood vessels entering and leaving tissue modeled by culture 26′. By way of elaboration and not limitation, model 10′ is provided in three layers L1, L2 and L3, each of which can be the same or different. Each layer of layers L1, L2, and L3 can comprise any embodiment of model 10 as described herein, including embodiments where a culture in present in each layer. However, as shown, layer L1 comprises a chamber 40 and a series of channels 42, 44, 46, and 48 configured for fluidic communication to receive inputs and/or outputs as shown by the arrows. Layer L3 comprises a chamber 50 and a series of channels 52, 54, 56, and 58 configured for fluidic communication to receive inputs and/or outputs as shown by the arrows. Layer L2 comprises culture 26′ and is configured similarly to the embodiment shown in FIG. 1C. Model 10′ comprises a microfluidic housing 12′, which comprises a cell culture chamber 14′ and one or more channels in communication with cell culture chamber 14′. The one or more channels comprise first channel 16′, second channel 18′, third channel 20′, and fourth channel 22′. Cell culture 26′ comprises zones Z1′ and Z2′, wherein patterned cells are provided. For example, zone Z1′ can comprise B cells and/or precursors thereof, and zone Z2′ can comprise T cells and/or precursors thereof. Zone Z3′ is a channel that defines a sinus that surrounds the cells of zone Z1′ and zone Z2′.

Continuing with reference to FIG. 10, fluidic communication between layers L1, L2, and L3, for example via one or more channels schematically represented by channel line CL. Communication can be provided in any direction along channel line CL as well as in the direction of the arrows in FIG. 10. Channel line CL passes through zones Z1′ and Z2′. Channel line CL can in some embodiments mimic blood flow and/or lymphatic fluid flow as needed. By way of example and not limitation, model 10′ is assembled either with irreversible bonding or with reversible methods such as clamping. A clamped model enables one to retrieve the culture for further analysis, e.g. by flow cytometry or high-magnification imaging.

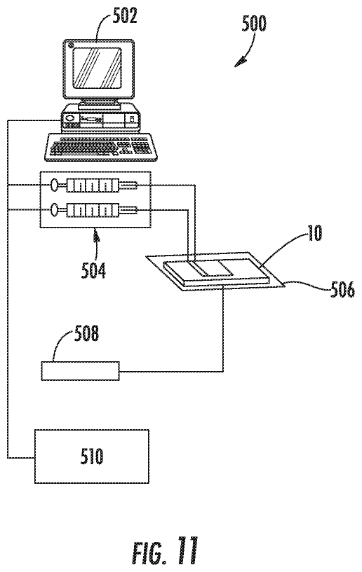

In vivo, flow is critical for chemotaxis, trafficking of cells, and delivery of chemical stimuli to and from the lymph node. Flow also plays a role in in vitro cell culture on a microchip and must be controlled to enable nutrient and waste exchange. Pressure-driven microfluidics (peristaltic pumps) can be used to drive fluid around and within the node, emulating lymphatic and interstitial flow. Channel architectures and flow rates are selected using computational fluid dynamics to mimic physiological lymphatic flow in the incoming lymphatic vessels (for example, 1-5 μm/s) and the interstitium (for example (0.1-10 μm/s). Referring now to FIG. 11, a representative embodiment of organ model system 500 is illustrated. System 500 generally comprises a computer 502 that can be used in controlling system 500, a reagent introduction device advantageously provided in the form of a pump assembly, generally designated 504, and model 10. Computer 502 is operatively connected to pump assembly 504 and can provide control of fluidic flow from pump assembly 504 to model 10. In some embodiments, pump assembly 504 comprises one or more peristaltic pumps and is in fluidic communication with the one or more channels for moving reagents into the one or more channels of model 10. For providing two or more reagents to model 10, pump assembly 504 comprises at least two or more pumps. Model 10 can be mounted on a microscope stage 506 typically provided with the microscope. In some embodiments, microscope stage 506 can be controllably actuated in X-Y or X-Y-Z space to align model 10 with pump assembly 504.

Continuing with reference to FIG. 11, system 500 comprises one or more other one or more other tissue-on-chip devices 508, wherein model 10 is in fluidic communication with the one or more other tissue-on-chip devices 508. Tissue-on-chip device 508 is also in fluidic communication with pump assembly 504 and computer 502 can control fluidic flow to go from pump assembly 504 to model 10 and/or to tissue-on-chip device 508. Computer 502 can also control the direction of fluidic flow to go from pump assembly 504 to model 10 then tissue-on-chip device 508 and vice versa. In some embodiments, the one or more other tissue-on-chip devices is selected from the group consisting of a liver device, a kidney device, a lung device, a brain device, and a tumor device. A representative such device is disclosed in Published U.S. Patent Application No. 2011/0086382 to Marx, published Apr. 14, 2011, incorporated herein by reference in its entirety.

Continuing with reference to FIG. 11, pump assembly 504 can be used in delivering a stimulus to system 500, such as by delivering a stimulus to model 10, delivering a stimulus to tissue-on-chip device 508, and combinations thereof In some embodiments, delivering the stimulus comprises delivering a vaccine to the model and/or system. In some embodiments, delivering the stimulus comprises delivering an established or new therapy or drug to the model and/or system. In some embodiments, delivering the stimulus comprises testing spatial organization of the model or system. In some embodiments, delivering the stimulus comprises varying a location and/or an inclusion of a particular cell type between a first model or system and a subsequent model and/or system, and/or varying a rate and/or distribution of fluidic flow through the model and/or system.

Continuing with reference to FIG. 11, system 500 comprises a readout device 510. Readout device 510 can be any suitable device that can be used in evaluating a response to a stimulus, such as but not limited to a microscope, an electrochemical array, a chromatography system, or an immunoassay system, and can be in electronic communication with computer 502 for transmission of control instructions, data, and the like. In some embodiments, evaluating a response to a stimulus comprises evaluating one or more of cell motility, gene expression, protein secretion, small molecule secretion, production of reactive oxygen species, and metabolic activity.

Thus, in some embodiments, the presently disclosed subject matter provides a microfluidic chip-based model of a tissue of an immune system. While not wishing to be limited to any particular theory of design, it should capture the essence of the tissue by mimicking the spatial organization of cells, matrix composition, and the patterns of fluid flow found in the tissue in vivo. In some embodiments, the core of the microfluidic chip-based model is a thick (100-300 μm), spatially patterned culture of cells and molecular cues suspended in a hydrogel matrix and enclosed within a microfluidic device. The thick hydrogel provides a 3D microenvironment for cell culture, while enabling simple imaging and chemical delivery. In some embodiments, it is formed within the microfluidic housing by sequential photopatterning as disclosed herein. In some embodiments, the microfluidic chip-based model is based on a blank microfluidic chamber and channels (see e.g., FIG. 6A (inlet)) that can be fabricated from an appropriate substrate material including but not limited to polydimethylsiloxane (PDMS) using by soft lithography or cyclic olefin copolymer (COC) using hot embossing.

Imprint lithography can also be employed for patterning a LN-chip of the presently disclosed subject matter. Imprint lithography is a hybrid patterning technique that combines embossing—patterning with a stamp or mold—with UV- or thermal-induced crosslinking of a liquid resist (Traub et al. (2016) Annu Rev Chem Biomol Eng 7:583-604). The method is conducted at room temperature and mild pressures, making it biocompatible. The advantage of the imprint method versus traditional photolithography is that an even delivery of photons across all the lateral features ensures well-controlled hydrogel curing times, thereby reducing the damage to cells (Tran & Nguyen (2017) J Sci Adv Mater Devices 2:1-14). Feature fidelity with vertical sidewalls is maintained due to use of an embossed soft stamp, rather than optically defined wall (Traub et al. (2016) Annu Rev Chem Biomol Eng 7:583-604). Furthermore, while bioprinting requires the squeezing of the cell-laden hydrogel fluid through a syringe, which can damage cells due to shear forces, cell patterning by imprinting occurs due to filling of the grooves on the stamp under capillary action. Hence, due to lower shear forces, cells may experience a lesser degree of damage during imprinting.

To generate a stamp that transmits high aspect ratio features with a high degree of fidelity, a high contrast resist, such as SU8 patterned by soft or hard contact photolithography (depending on SU8 thickness), will be used as a master to emboss on a COC (cyclic olefin copolymer) substrate (Glinsner et al. (2010) Microelectron Eng 87:1037-1040). The stamp can be functionalized with a fluorinated silane to minimize adhesion to the hydrogel. Next, the stamp can be loaded with the liquid resist, such as but not limited to the hydrogel GelMA containing the cells of interest. GelMA can be dispensed to fill the grooves by capillary action. Pressure can be applied on the opposite side of the hydrogel layer using a planar glass plate that has been pre-treated with methacrylated silane to anchor the GelMA to the surface. Exposure to UV can then enable curing of the hydrogel with the cells. It is noted that hydrogels other than GelMA can be employed, including but not limited to GelNB, provided that the hydrogel of choice can be photocrosslinked.

A possible disadvantage of the imprint method is the presence of a residual layer on the back side of the hydrogel, thereby leaving a thin layer wherein the cells are not patterned. To overcome this, two strategies can be employed. First, an EVG 620 tool (EV Group, Inc., Albany, N.Y., United States of America) cab be configured to allow for gradually increasing applied pressure to the back side of the hydrogel layer, slowly squeezing down the residual layer prior to UV curing to a thickness of <1 μm, thereby excluding cells from the residual layer. In some embodiments, this procedure enables exclusion of cells without crushing them. A second strategy can involve patterning a resist layer on the back side of the glass plate, so that UV exposure is limited only to those regions where the cells of interest are to be patterned and a post-exposure wash removes the hydrogel from uncured areas. The distance from the resist to the patterned hydrogel can be minimized by using a very thin glass plate; this reduces the gap wherein bending of light around features can occur. Adding a photomask to the glass plate (depicted in FIG. 6B) has an added advantage during multi-step patterning, because only the newly imprinted features is exposed to UV light at each step, thus preventing damage to fragile cells from repeated UV-exposure.

In some embodiments the tissue is a lymph node. In some embodiments, the tissue is spleen. In some embodiments, a length scale model of a mammalian lymph node is provided. In some embodiments, the lymph node is selected from the group including but not limited to a skin-draining lymph node, a cervical lymph node, a mesenteric lymph node, an iliac lymph node, a mediastinal lymph node, a popliteal lymph node, a tonsil, a Peyer's patch, and a tertiary lymphoid structure that forms in sites of chronic inflammation.

In some embodiments, the model comprises a microfluidic housing comprising a cell culture chamber and one or more channels in communication with the cell culture chamber; and a cell culture residing in the cell culture chamber, the cell culture comprising one or more cells of the immune system. In some embodiments, the cell culture is a three-dimensional (3D) cell culture.

In some embodiments, the one or more cells of the immune system are patterned within the cell culture. As discussed in more detail elsewhere herein, in some embodiments, the one or more cells of the immune system are patterned in one or more zones. In some embodiments, one or more channels pass through one or more zones. See e.g., FIGS. 9A and 9B. In some embodiments, one or more channels surround the one or more zones. See e.g., FIGS. 4A-4D. In some embodiments, a first zone comprising a first type of the one or more cells of the immune system is surrounded by a second zone of a second type of the one or more cells of the immune system. In some embodiments, the first type of the one or more cells of the immune system is B cells and the second type of the one or more cells of the immune system is T cells. In some embodiments, the first zone and the second zone are surrounded by a third zone, which can be a channel or a sinus. Representative approaches for patterning the cells are disclosed elsewhere herein, including above with respect to the discussion of the Figures. Additional representative approaches are also described in the Examples presented herein below.

In some embodiments, the one or more cells of the immune system can be healthy and/or naive (resting) cells of the immune system. In some embodiments, the primary immune cells are B cells, T cells, dendritic cells, macrophages, and/or neutrophils. In some embodiments, non-immune stromal cells are also included within the cell culture. In some embodiments, one or more neurons are included within the cell culture. In some embodiments, the cells are genetically modified cells. By way of example and not limitation, the genetically modified cells can be obtained from a genetically modified animal and/or can be cells modified by any suitable genetic modification technique as would be apparent to one of ordinary skill in the art, such as but not limited to a CRISPR/Cas9 technique. In some embodiments, the one or more cells of the immune system can be diseased cells, such as cells isolated from a diseased subject or cells modified in vitro and/or ex vivo to recapitulate a disease state. In some embodiments, the one or more cells of the immune system can be activated cells. By way of example and not limitation, the activated cell can be a cell collected from a donor that had has been treated with a drug or other therapy, or a donor that is a disease model, or a cell that was treated in vitro with a drug or therapy. The other therapy can be any suitable therapy as one be apparent to one of ordinary skill in the art upon a review of the instant disclosure, including but not limited to a vaccine therapy. Any combination of the aforementioned cells can be also be employed.

In some embodiments, the one or more cells of the immune system are derived from a murine or human source, although any suitable source as would be apparent to one of ordinary skill in the art upon a review of the present disclosure can be employed, including other mammalian subjects and other vertebrate subjects. In some embodiments, the one or more cells of the immune system are derived from stem cells, such as but not limited to induced pluripotent stem cells. In some embodiments, the one or more cells of the immune system are derived from a diseased donor.

In some embodiments, the cell culture comprises one or more non-immune cell type. Any suitable non-immune cell type can be employed as would be apparent to one of ordinary skill in the art upon a review of the present disclosure, such as but not limited to stromal cells, endothelial cells, and/or neural cells (such as but not limited to neurons).

In some embodiments, the cell culture comprises a scaffold composition. In some embodiments, the cell culture is provided by a precursor composition, which in some embodiments comprises one or more cells, or cell types, and a scaffold-forming composition. In some embodiments the precursor composition comprises a reagent that induces pattern formation, in some embodiments, the scaffold-forming composition comprises a reagent that induces pattern formation. For example, the reagent that induces pattern formation can comprise a photo-reactive crosslinking group and/or a photoinitiator. In some embodiments, the photo-reactive crosslinking group and the photoinitiator are included in a hydrogel that also comprises cells for which deposition and/or patterning on an LN-chip of the presently disclosed subject matter is desired. In some embodiments, the hydrogel is a biocompatible photo-crosslinkable hydrogel such as, but not limited to a gelatin methacrylate hydrogel (GelMA) or a modular gelatin-norbornene (GelNB) hydrogel. In some embodiments, a photoinitiator is selected from the group consisting of lithium phenyl-2,4,6-trimethylbenzoylphosphinate (LAP; see e.g., U.S. Pat. No. 9,402,710) and members of the Irgacure family of photoinitiators, including but not limited to 2-hydroxy-2-methylpropiophenone (CAS No. 7473-98-5; also known as Irgacure 1173). In some embodiments the scaffold-forming composition comprises a protein-based biomaterial, further optionally a gelatin methacrylate, a gelatin norbornene, albumin, and/or a collagen. In some embodiments, the scaffold-forming composition comprises a non-protein material, such as but not limited to polyethylene glycol.

In some embodiments, the microfluidic housing comprises one or more surfaces having a surface chemistry modification. Thus, in some embodiments, the reagent that induces pattern formation can comprise a reagent that interacts with a surface chemistry modification, such via a covalent or ionic bond, or other interaction.

In some embodiments, the precursor composition and/or the scaffold-forming composition can comprise additional components. For example, in some embodiments heparin is included in the precursor composition and/or the scaffold-forming composition. The heparin serves to bind proteins called chemokines, which are secreted by the cells. The chemokines can also be added deliberately to precursor composition and/or to the scaffold-forming composition, to add additional functionality. Other reagents besides heparin and chemokines could also be included in the precursor composition and/or scaffold-forming composition, such as but not limited cell-binding moieties, receptor ligands, inhibitors, enzymes, or drugs. In some embodiments, the cell culture comprises a mixture of scaffolds. In some embodiments, the scaffolds are dispersed homogeneously through the cell culture and/or are patterned in one or more different zones of the cell culture.

In some embodiments, the microfluidic housing comprises a material selected from the group including but not limited to a silicone rubber (optionally polydimethyl siloxane, PDMS), glass, a thermoplastic polymer, a three-dimensional (3D) printed resin, a biomaterial (optionally a PEG-based material or a protein-based material), and any combination thereof.

In some embodiments, the microfluidic housing comprises one or more surfaces having a surface chemistry modification. A representative surface chemistry modification includes but are not limited to 3-(Trimethoxylsilyl)propyl methacrylate (TMSPMA), for PDMS, which allows GelMA to covalently bind to glass. Alternatively, a thiolated silane can be employed for the same purpose when gelatin norbornene (GelNB) is employed. However, alternative strategies for modifying surface chemistries include, but are not limited to coating the surface with a protein or a mixture of proteins, such as but not limited to coating the surface with serum albumin and/or pretreating the surface with culture media that contains serum. Patterned structures will withstand incoming fluid flow. In some embodiments, this treatment involves a treatment time of about 15 minutes and includes adding a PDMS Mask in the area on the housing for the cell culture chamber and plasma cleaning. This provides a surface ready for ready for irreversible bonding. Another representative surface chemistry modification, this one for glass, includes but is not limited to functionalization with silane polyethylene (glycol) methacrylate. This allows GelMA to covalently bind to surface, prevents non-specific adhesion, and provides a slightly hydrophilic surface. In some embodiments, this treatment involves a treatment time of about 40 minutes and includes adding a PDMS Mask in the area on the housing for the cell culture chamber and plasma cleaning. This provides a surface ready for ready for irreversible bonding. As additionally described elsewhere herein, in some embodiments, the scaffold-forming composition comprises a reagent that interacts with a surface chemistry modification, such via a covalent or ionic bond, or other interaction. Thus, any suitable combination of surface chemistry modification and interacting reagent as would be apparent to one of ordinary skill in upon a review of the present disclosure is provided in accordance with the presently disclosed subject matter.

In some embodiments, the scaffold can optionally contain additional structural elements such as fibers and/or particles at the nano and/or microscale, which in some embodiments can provide support for, facilitate, or otherwise enhance migration of cells within the cell culture. By way of example and not limitation, an electrospun fiber can be employed as part of the cell culture. Electrospinning can be performed essentially as set forth in U.S. Pat. No. 8,586,345 (see also Russell & Lampe (2017) ACS Biomater Sci Eng 3:3459-3468). Alternatively or in addition, the SPRAYBASE© suite of electrospinning emitters and collectors can be employed as per the manufacturer's basic protocols (SPRAYBASE®, Cambridge, Mass., United States of America). By way of further example and not limitation, collagen can be electrospun out of a solution in 1,1,1,3,3,3 hexafluoro-2-propanol, a volatile solvent that is easily driven off (Matthews et al. (2002) Biomacromolecules 3:232-238). Similarly, PCL/gelatin can be spun out of a solution in trifluoroethanol (Zhang et al. (2005) J Biomed Mater Res B Appl Biomater 72B:156-165; Alvarez-Perez et al. (2010) Biomacromolecules 11:2238-2246). Fibers (not containing cells) can be spun into an unaligned mat (sterilized by soaking in 70% ethanol for 30 minutes, then rinsed extensively with saline) to mimic the stromal network found in vivo (Ushiki (2002) Arch Histol Cytol 65:109-126). The fiber mat can then be embedded in a cell-loaded GelMA-based scaffold-forming precursor composition, which can be gelled using photolithography as described herein. Cell viability in the bulk hydrogel can be tested by staining with Calcein and PI to confirm that the addition of fibers does not reduce viability.

In addition to photolithography and stamping, bio-printing can also be employed to pattern an LN-chip of the presently disclosed subject matter. By way of example and not limitation, the 3DDISCOVERY Bioprinter (regenHU Ltd., Villaz-St-Pierre, Switzerland) for direct patterning of cells in gel onto the microchip surface (see e.g., U.S. Patent Application Publication No. US 2017/0368225). In some embodiments, the microfluidic housing comprises at least two channels in communication with the cell culture chamber. In some embodiments, at least one of the channels is configured to deliver input to the cell culture chamber and at least one of the channels is configured to receive output from the cell culture chamber. In some embodiments, the microfluidic housing comprises one or more additional chambers in communication with the cell culture chamber. In this case, in some embodiments, two or more channels are in communication with the cell culture chamber, where one or more of the channels is configured to deliver input to the cell culture chamber from the one or more additional chambers and/or one or more of the channels is configured to receive deliver output from the cell culture chamber to the one or more additional chambers. See e.g., FIG. 10.

In some embodiments, the one or more channels in communication with the cell culture chamber comprise one or more channels configured to deliver input to one or more zones in the cell culture. In some embodiments, a first zone comprising a first type of the one or more cells of the immune system is surrounded by a second zone of a second type of the one or more cells of the immune system. In some embodiments, the first type of the one or more cells of the immune system is B cells and the second type of the one or more cells of the immune system is T cells. In some embodiments, the first zone and the second zone are surrounded by a third zone, which can be a channel or sinus. See e.g., FIGS. 4A-4D and 9A-9B.

In some embodiments, the microfluidic housing comprises one or more additional layers comprising one or more additional channels and/or one or more additional chambers. See e.g., FIG. 10. In some embodiments, the additional layers are disposed above and/or below a first layer comprising the cell culture chamber and the one or more channels in fluidic communication with the cell culture chamber. In some embodiments, the one or more additional channels and/or one or more additional chambers are in fluidic communication with the cell culture chamber and the one or more channels in fluidic communication with the cell culture chamber of the first layer. In some embodiment, the fluidic communication is achieved via a valve, a port, and/or a semi-permeable membrane between the layers.

In some embodiments, the model including the microfluidic housing is assembled either with irreversible bonding or with reversible methods such as clamping. A clamped model enables one to retrieve the gel for further analysis, e.g. by flow cytometry or high-magnification imaging.

In some embodiments, one or more pumps is externally disposed relative to the model. In some embodiments, the one or more pumps is in fluidic communication with the one or more channels for moving reagents into and through the one or more channels. See e.g., FIG. 11. In some embodiments, a system comprising the model and one or more other tissue-on-chip devices is provided, wherein the model is in fluidic communication with the one or more other tissue-on-chip devices. See e.g., FIG. 11. In some embodiments the one or more other tissue-on-chip devices is selected from the group comprising but not limited to a liver device, a kidney device, a lung device, a brain device, and a tumor device.

In some embodiments, a method for patterning cells in a culture on a microfluidic chip is provided. In some embodiments, the presently disclosed methods comprise: providing a microfluidic housing comprising a cell culture chamber and one or more channels in communication with the cell culture chamber; and patterning cells in the cell culture chamber to provide a culture in the cell culture chamber. In some embodiments, the cells comprise one or more cells of the immune system. Thus, the presently disclosed methods provide for the preparation and/or production of a microfluidic chip-based model of a tissue of an immune system in accordance with the presently disclosed subject matter. In some embodiments, the culture is a three-dimensional (3D) culture.

In some embodiments, the one or more cells of the immune system are patterned within the cell culture. As discussed herein above, in some embodiments, the one or more cells of the immune system are patterned in one or more zones. In some embodiments, one or more channels pass through one or more zones. See e.g., FIGS. 9A and 9B. In some embodiments, one or more channels surround the one or more zones. See e.g., FIGS. 4A-4D. In some embodiments, a first zone comprising a first type of the one or more cells of the immune system is surrounded by a second zone of a second type of the one or more cells of the immune system. In some embodiments, the first type of the one or more cells of the immune system is B cells and the second type of the one or more cells of the immune system is T cells. In some embodiments, the first zone and the second zone are surrounded by a third zone, which can be a channel or a sinus.

In some embodiments, the microfluidic housing comprises a material selected from the group including but not limited to a silicone rubber (optionally polydimethyl siloxane, PDMS), glass, a thermoplastic polymer, a three-dimensional (3D) printed resin, a biomaterial (optionally a PEG-based material or a protein-based material), and any combination thereof.

In some embodiments, the patterning comprises directing a precursor composition comprising one or more cells of the immune system to the cell culture chamber through the one or more channels. In some embodiment, the precursor composition comprises a reagent that induces pattern formation, in some embodiments, the precursor composition comprises a scaffold-forming composition, which can comprise a reagent that induces pattern formation. In some embodiments, the precursor composition comprises one or more non-immune cell types, such as but not limited stromal cells, endothelial cells, and/or neural cells such as but not limited to neurons.

In some embodiments, the patterning comprises directing light through a photomask covering a portion of the microfluidic housing, providing one or more architectural features in the cell culture chamber, modifying surface chemistry of the microfluidic housing, and any combination thereof. In some embodiments, the light directed through the photomask has a wavelength ranging from about 300 nanometers (nm) to about 800 nm, including but not limited to 405 nm. In some embodiments, the photomask is selected from the group comprising but not limited to a printed transparency mask, a chrome mask, and a digital mask, and another standard mask used for photolithography.

In some embodiments, the patterning comprises altering the surface chemistry of the microfluidic housing and the reagent that induces pattern formation comprises a moiety that reacts with the altered surface chemistry. Thus, in some embodiments, the microfluidic housing comprises one or more surfaces having a surface chemistry modification. A representative surface chemistry modification includes but are not limited to 3-(Trimethoxylsilyl)propyl methacrylate (TMSPMA), for PDMS, which allows GelMA to covalently bind to glass. Patterned structures will withstand incoming fluid flow. In some embodiments, this treatment involves a treatment time of about 15 min and includes adding a PDMS Mask in the area on the housing for the cell culture chamber and plasma cleaning. This provides a surface ready for ready for irreversible bonding. Another representative surface chemistry modification, this one for glass, includes but is not limited to functionalization with silane polyethylene (glycol) methacrylate. This allows GelMA to covalently bind to surface, prevents non-specific adhesion, and provides a slightly hydrophilic surface. In some embodiments, this treatment involves a treatment time of about 40 min and includes adding a PDMS Mask in the area on the housing for the cell culture chamber and plasma cleaning. This provides a surface ready for ready for irreversible bonding. As additionally described elsewhere herein, in some embodiments, the scaffold-forming composition comprises or forms a group that interacts with a surface chemistry modification, such via a covalent or ionic bond, or other interaction. Thus, any suitable combination of surface chemistry modification and interacting reagent as would be apparent to one of ordinary skill in upon a review of the present disclosure is provided in accordance with the presently disclosed subject matter.

In some embodiments, the model including the microfluidic housing is assembled either with irreversible bonding or with reversible methods such as clamping. A clamped model enables one to retrieve the gel for further analysis, e.g. by flow cytometry or high-magnification imaging.

In vivo, fluidic flow is critical for chemotaxis, trafficking of cells, and delivery of chemical stimuli to and from the lymph node (Swartz (2001) Adv Drug Deliv Rev 50:3-20). Fluidic flow should thus be carefully controlled to enable nutrient and waste exchange (Regehr et al. (2009) Lab Chip 9:2132-2139; Bonvin et al. (2010) Biotechnol Bioeng 105:982-991; van Duinen et al. (2015) Curr Opin Biotechnol 35:118-126). In some embodiments, pressure-driven microfluidics (peristaltic pumps) drive fluid around and within the LN-chip, emulating lymphatic and interstitial flow (see e.g., FIG. 11). In some embodiments, channel architectures and fluidic flow rates can be selected using computational fluid dynamics to mimic physiological lymphatic flow in the incoming lymphatic vessels (1-5 μm/s) and the interstitium (0.1-10 μm/s; Tomei et al. (2009) J Immunol 183:4273-4283; Munson & Shieh (2014) Cancer Manag Res 6:317-328; Jafarnej ad et al. (2015) Lymphat Res Biol 13:234-247). In some embodiments, the LN-chip can comprise one or more additional layers of channels beneath the culture chamber, which in some embodiments can comprise ports passing upward to model blood vessels entering and leaving the tissue.

For example, interstitial flow can be employed to deliver oxygen and nutrients to sustain viability in the center of the hydrogel, meaning that adequate oxygenation can be required. If, for example, cells on the LN-chip die, adequate oxygenation can be tested, and one or more of faster flow, the addition of one or more transverse sinuses (channels) through the 3D culture, or both can be used to increase access to nutrients. (2) Activated T cells are supported by autocrine and paracrine IL-2 secretion (Besser et al. (2009) Cytotherapy 11:206-217). If viability is preserved but inadequate T cell function is present in long-term culture, supplementation with IL-2 can be employed (50-600 U/mL; Besser et al. (2009) Cytotherapy 11:206-217; Au-Yeung et al. (2017) J Immunol 198:2445-2456).

In some embodiments, a method for modeling an immune response of a subject is provided in accordance with the presently disclosed subject matter. In some embodiments, the method comprises providing a model of a tissue of the immune system in accordance with the presently disclosed subject matter or a system according the presently disclosed subject matter; delivering a stimulus to the model; and evaluating a response to the stimulus.

In some embodiments, delivering the stimulus comprises delivering a vaccine to the model or system; and evaluating the response comprises evaluating one or more cell motility, gene expression, protein secretion, small molecule secretion, production of reactive oxygen species, and metabolic activity. In some embodiments, delivering the stimulus comprises delivering an established or new therapy or drug to the model or system. In some embodiments, delivering the stimulus comprises testing spatial organization of the model or system, such as but not limited to delivering the stimulus comprises varying a location and/or an inclusion of a particular cell type between a first model or system and a subsequent model or system, and/or varying a rate and/or distribution of fluid flow through the model or system.

As such, basic cellular function can be optimized using T cell motility as an indicator of the state and health of the tissue. At rest, T cells follow a modified random walk with ˜10 μm/min average velocity (depicted in FIGS. 4A and 4C), which slows to 1-4 μm/min upon activation or inflammation of the tissue (Worbs & Förster (2009) in: Dustin & McGavern (eds.) Visualizing Immunity. Springer Berlin Heidelberg; pages 71-105). T cell motility after 0, 1, 4, and 7 days of culture in the microfluidic LN-chip can be measured and compared to that of fresh, healthy lymph node tissue ex vivo. Time-lapse imaging of the LN-chip and the tissue slices can be conducted using a confocal microscope or a two-photon microscope. Cell traces can be generated from image analysis using Matlab software available from The MathWorks, Inc. of Natick, Mass., United States of America in conjunction with the open-source software Cell Tracker (Piccinini et al. (2016) Bioinformatics 32:955-957).

In some embodiments, the model is patterned using healthy and/or naïve (resting) cells to model a healthy tissue of the immune system. In some embodiments, the model is patterned with cells from a diseased donor to model that disease. In some embodiments, the model is patterned with genetically modified cells to model a disease and/or to test a hypothesis about a role of a modified gene or pathway.

### EXAMPLES

The following Examples provide illustrative embodiments. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Exemplary LN-chip Device

An exemplary microfluidic device that contained a chamber housing a 3D patterned cell culture via UV photo-crosslinkable hydrogels was produced. It is depicted in FIG. 2A. Referred to herein as an “LN-chip”, the LN-chip device 10 comprised two microfluidics “afferent lymphatic” channels 16 and 18, each with two inlet channels 16a and 16b, a central culture chamber 14, two air release channels 30 and 32, and one microfluidic “efferent lymphatic” channel 22. (see FIG. 2A). The device 10 comprised a polydimethylsiloxane (PDMS) layer and a glass layer. The surfaces of both layers were treated individually in order to provide the correct surface chemistry compatible with the materials it housed. The glass layer was treated with 20 mg/ml silane polyethylene glycol acrylate (PEGDA, 5000 MW) in a 95% ethanol/5% water solution. The PDMS was treated with a mixture of 3-(trimethoxysilyl)propyl methacrylate and Trichloro(1H,1H,2H,2H-perfluorooctyl)silane. The two layers were then irreversibly bonded via plasma bonding. The chamber area was protected with PDMS “masks” during the plasma exposure to keep the desired surface modifications intact. The actual device is shown in FIG. 2B.

### Example 2

### Preliminary Results for Synthesis of GelMA and its Optimization as a Culture Matrix for the LN-chip

In initial experiments, GelMA was produced with a “medium” degree of functionalization by replacing free amines with methacryloyl groups. The degree of functionalization was assessed by using a ninhydryn assay, which qualitatively measured the number of free amine groups present in solution. Approximately 55% of available amines were functionalized. Different concentrations of GelMA were tested and the stiffness of the resulting gel was measured as a function of exposure time. The 8% GelMA, 0.10% w/v LAP, 4-minute exposure condition matched the reported stiffness of lymphoid tissue (i.e., 1.5-3 kPa). Cellular viability in the GelMA matrix (measured by the ratio of Calcein AM to Propidium Iodide) was not significantly diminished after 1 or 4 minutes of UV exposure.

### Example 3

### Quantification of Cells Appearing in “Wrong” Zones

An exemplary LN-chip of the presently disclosed subject matter was successfully photopatterned in gelatin methacrylate (GelMA) inside a PDMS chamber bonded to a glass coverslip. The interior was pre-treated with methacrylated silane to facilitate covalent bonding to GelMA. A sheet of polystyrene was placed over the PDMS to reduce oxygen diffusion and enable photo-polymerization (see Dendukuri et al. (2008) Macromolecules 41:8547-8556). The LN-chip was filled with B cell precursor solution as described herein, exposed through a photomask (see e.g., FIG. 6B) to pattern the B cell zone (see e.g., FIG. 6C and 6D; “B”) , and rinsed with buffer to reveal the follicles in the B cell zone. The LN-chip was filled with T cell precursor solution as described herein, exposed through a photomask to pattern the T cell zone (see e.g., FIGS. 6C and 6D; “T”) , and rinsed with buffer to create the surrounding T cell zone as described herein.

The patterning of the B and T cell zones were carried out at 25° C., but undesirable adhesion of B cells in the T cell zone was observed. The patterning of the B and T cell zones was repeated but washing with buffer at 4° C. was performed after the B cell zone was established. The cold buffer wash reduced non-specific cell adhesion at densities as high as 10×10⁶ cells/mL (see FIG. 6E).

### Example 4

### Testing of Various Crosslinking Conditions

The light dose (intensity×time) and concentration of gelatin precursor can be varied and the stiffness measured on a rheometer until it matches the stiffness of lymphoid tissue (0.12-3 kPa; Purwada et al. (2015) Biomaterials 63:24-34; de la Zerda et al. (2018) APL Bioeng 2:021501) within two-fold. Variability is expected between batches of GelMA/GelNB, thus the degree of functionalization can be measured and stiffness for each can be optimized. Unfractionated human WBCs can be suspended in the precursor solution at medium density (10⁷ cells/mL) and gelled off-chip at 405 nm to determine the exposure intensity and time that optimize cell viability at 1 and 24 hour after patterning.

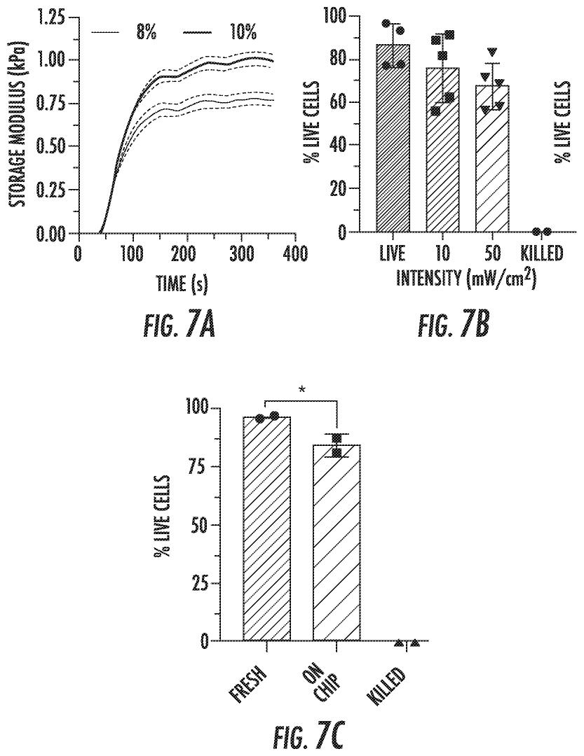

In preliminary experiments, 8% and 10% GelMA were synthesized by replacing free amines with methacryloyl groups, and ˜30% functionalization was obtained according to a ninhydrin assay (Levett et al. (2014) Acta Biomater 10:214-223; Loessner et al. (2016) Nat Protoc 11:727-746). The results are presented in FIG. 7A. Rheology indicated that 10% GelMA with 0.10% w/v LAP, exposed to 2700 mJ/cm², averaged ˜1 kPa, matching the reported stiffness of lymphoid tissue (Purwada et al. (2015) Biomaterials 63:24-34). At this dose, viability of murine splenocytes 1 hour after off-chip gelation was within 20% of fresh cells for intensity as high as 50 mW/cm² (see FIG. 7B). FIG. 7C shows the viability assessed by Calcein AM/PI staining after mixed splenocytes were patterned on chip and left to rest for one hour with constant perfusion of media at 0.2 μL/min.

### Example 5

### Modeling Fluidic Flow Rates Through the LN-chip

Initially, fluidic flow rates through the LN-Chips of the presently disclosed subject matter were predicted to be physiological. A 2D simulation of fluidic flow through a 10-mm LN-chip, conducted with the finite element method of COMSOL MULTIPHYSICS® software (COMSOL, Inc., Burlington, Massachusetts, United States of America). The tissue was modeled as a porous matrix and the lymphatics and sinus as open channels. Incoming flow velocity through the afferent lymphatics was set to 5 μm/second (Roadmap Epigenomics Consortium, Kundaje et al. (2015) Integrative analysis of 111 reference human epigenomes. Nature 518:317-330).

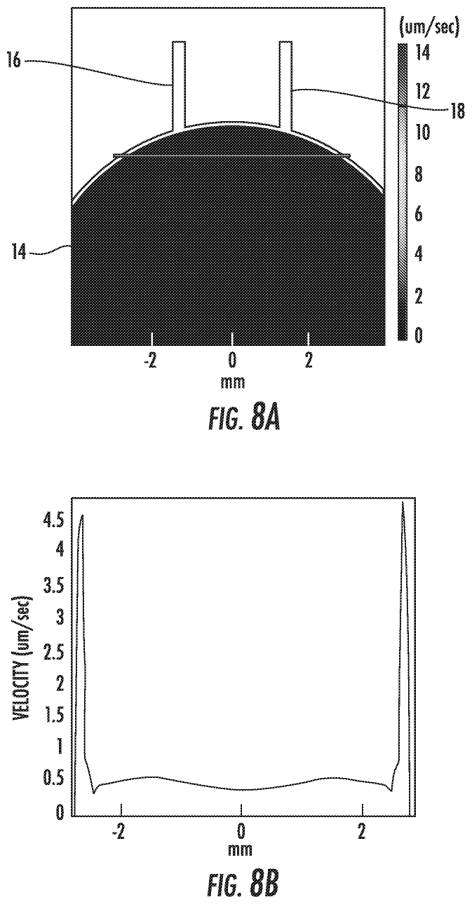

The results are summarized in FIGS. 8A and 8B. FIG. 8A is a plot showing predicted velocity (μm/s). Flow within the channels 16 and 18 and in the sinus Z3 (lighter area surrounding the culture chamber are shown to be about 4-5 μm/second, whereas flow within the culture chamber 14 was predicted to be lower (less than 1 μm/second). FIG. 8B provides a velocity profile along the dotted line in FIG. 8A, which would correspond to the B cell region. The predicted interstitial flow velocity was about 0.5 μm/sec in the hydrogel and 4.5 μm/s in the sinus, matching physiological values (Farh et al. (2015) Nature 518:337-343).

### Example 6

### Modelling Inflammation and Adaptive Immunity

An exemplary LN-chip is stimulated with microbial signals such as bacterial DNA (CpG) and lipopolysaccharide (LPS) to model inflammation. Read-outs include the rates and direction of cell migration, metabolic activity, and cytokine secretion (e.g. ,IL-1β, IL-6, TNP-α).

To model adaptive immunity, on-chip “vaccination” is performed by delivering a model antigen, the protein chicken egg ovalbumin (OVA), plus LPS or CpG (as an adjuvant), through the lymphatic channels of an exemplary LN-chip. The chip is preloaded with naïve T cells that are specific to OVA, called OTII (CD4⁺) and OTI (CD8⁺) cells. Readouts after vaccination include cell migration, metabolic activity, and cytokine secretion (those above plus IFN-γ and IL-2), plus proliferation of OVA-specific T cells and antigen-specific antibody secretion.

### Example 7

### Modelling Chronic Inflammation and Autoimmunity

Chronic inflammation and autoimmunity remain some of the most difficult diseases to treat. Disease-specific versions of the LN-chip provide a first-in-class tool to rapidly test new therapies and hypotheses for chronic inflammation. (Leleux et al. (2015) J Controlled Release 219:610-621)

MS is used as a case study for immune-mediated chronic inflammation. MS is characterized by repeated waves of neuroinflammation that destroy the myelin sheath and eventually axons, eventually causing cognitive deficit and total paralysis. Myelin-specific T cells are thought to be activated in the brain-draining cervical lymph nodes. To model this process, a strategy similar to the generation of experimental autoimmune encephalomyelitis (EAE), the in vivo mouse model of MS (McCarthy et al. (2012) Methods Mol Biol 900:381-401) is followed. An LN-chip is preloaded with T cells specific to myelin (generated ex vivo) and vaccinated with myelin peptides. The number and positions of patterned myelin-specific cells and the dosage and timing of myelin challenge is varied until the chip replicates benchmark readouts from slices from EAE mice: decreased T cell motility, increased proliferation, myelin-specific cytokine secretion, and antibody production. Cervical nodes from EAE mice are also examined.

After initial inflammation resolves, a “relapse” is modeled by delivering the same myelin peptides through the lymphatic channels of an exemplary LN-chip. The dose and timing of delivered antigen is varied until the inflammatory response matches that of an EAE lymph node slice. Inflamed lymph nodes are significantly enlarged and reorganized compared to naive nodes. If it is observed that the “naïve” LN-chip does not recapitulate the inflammatory dynamics observed in LN from EAE mice, the LN-chip is redesigned with respect to one or more of size, lymphatic structure, and cellular arrangements to emulate an inflamed state. To do so, the spatial distributions of lymphatics, blood vessels, and cells (DC subsets, CD4+ and CD8+ T cells, and B cells) in EAE cervical nodes are characterized and translated to the LN-chip.

### Example 8

### Surface Treatment to Reduce Cracking

Under certain fabrication conditions, the patterned LN-chips had visible lines in them as a result of being casted onto non-smooth surfaces. The treatment of the surface was optimized to prevent these cracks from appearing. Particularly, both the PDMS and glass pieces were plasma-treated for only 20 seconds as opposed to the 80 seconds used previously. The pieces were then pressed together and placed in 120° C. oven for 20 minutes to bond them. The shortened plasma treatment prevented the cracking of the PDMS. Both layers were also treated for 2 hours with 3-(trimethoxylsilyl) propyl methacrylate (TMSPMA) via vapor deposition after the plasma bonding. This allowed the methacrylate groups in the patterned cultures to anchor onto both surfaces of the chip.

### Example 9

### Exemplary Loading and Rinsing Protocol

It had also been observed that when introducing samples containing a high density of cells, it was difficult to achieve target patterns without obtaining non-specific cell adhesion. For example, it was frequently observed that about 30% of cells in the outer T cell zone were actually B cells that were supposed to be localized solely in the inner B cell zone. To solve this problem, the protocol for loading cells into the chip was optimized by varying cell density and buffer temperature.

Non-specific adhesion increased with higher cell densities independent of buffer temperature. However, by decreasing the temperature of the cell suspension to 4° C., non-specific adhesion dropped to below 15% for cell densities of 1-10×10⁶/mL.

### Example 10

### Cell Viability as Function of Light Intensity

One of the potential disadvantages of photo-patterning primary cells is the potential damage caused by the light irradiation. To assess this, the effect that varying light intensity had on cells was tested. Cells were exposed at various intensities of light while keeping the dose constant (intensity×time=1970 mW/cm²). No statistical difference was observed between the percentage of live cells exposed to 10 mW/cm² or 50 mW/cm², or among the unexposed control and the cells exposed to 10 mW/cm² or 50 mW/cm².

### Example 11

### Viability After Patterning On-chip

It was possible to maintain high cell viability post-patterning while having media perfusion. These results showed the quantified viability (percentage of live cells) of patterned mixed splenocytes, one-hour after being patterned on the chip, under constant fluid flow at 0.2 μL/min. The patterned cultures retained high viability, within 15% that of unexposed “fresh” cells. These were at 10×10⁶ cells/mL and were given a light dose of 1970 mJ/cm².

### Discussion of the Example

Despite a growing catalogue of the actors in immunity, there are still no cures and few effective long-term treatments for chronic inflammatory disease. Disclosed herein are method and devices that are designed to fill in a critical gap of where and when molecules and cells respond to inflammatory events, thus advancing mechanistic research of immune dynamics in living systems. Building the first functional microfluidic model of a lymph node advances knowledge in both its approach and application: (1) Combining spatially patterned 3D cultures with microfluidics required technical advances that can advance the national organ-on-chip effort. This work has generated innovative combinations of UV-photolithography, 3D printing, nano-imprint lithography, and fluidic-flow-based patterning that can be useful for any spatially organized or hierarchical tissue models. (2) This effort has created the first spatially structured model of a lymph node. Experiments testing immune function after structural rearrangement or variation in lymphatic flow rates are challenging or impossible in vivo, but are straightforward to perform on the chip. Thus, experiments on the LN chip can provide fundamental insight into the cellular and molecular interactions that are essential to adaptive immunity. Furthermore, the LN-chip of the presently disclosed subject matter can be integrated to with models of brain, gut, and tumors to enable an entirely new type of study of whole-body immune responses and multi-organ diseases. In some embodiments, the LN-chip can also be populated with human cells to generate a platform for rapid drug screening for new immunotherapies. This is essential for national priorities such as cancer, Alzheimer's disease, and chronic autoimmunity.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A microfluidic chip-based model of a tissue of an immune system, comprising:
(a) a microfluidic housing comprising a cell culture chamber and one or more channels in communication with the cell culture chamber; and
(b) a cell culture residing in the cell culture chamber, the cell culture comprising one or more cells of the immune system.

2. The model of claim 1, wherein the cell culture comprises a three-dimensional (3D) cell culture.

3. The model of claim 1 or claim 2, wherein the one or more cells of the immune system are patterned within the cell culture.

4. The model of any one of claims 1-3, wherein the one or more cells of the immune system comprise:
(a) healthy and/or naive (resting) cells of the immune system;
(b) primary immune cells, optionally wherein the primary immune cells are B cells, T cells, dendritic cells, macrophages, and/or neutrophils;
(c) genetically modified cells, optionally cells obtained from a genetically modified animal and/or cells modified by a CRISPR/Cas9 method;
(d) a diseased cell;
(e) an activated cell, optionally a cell collected from a donor that has been treated with a drug or other therapy, or a donor that is a disease model, or a cell that was treated in vitro with a drug or therapy; or
(e) any combination thereof.

5. The model of any one of claims 1-4, wherein the one or more cells of the immune system are derived from a murine or human source; wherein the one or more cells of the immune system are derived from stem cells, optionally induced pluripotent stem cells; and/or wherein the one or more cells of the immune system are derived from a diseased donor.

6. The model of any one of claims 1-5, wherein the one or more cells of the immune system are patterned in one or more zones, optionally wherein one or more channels pass through the one or more zones and/or wherein one or more channels surround the one or more zones.

7. The model of claim 6, wherein a first zone comprising a first type of the one or more cells of the immune system is surrounded by a second zone of a second type of the one or more cells of the immune system, optionally wherein the first type of the one or more cells of the immune system is B cells and the second type of the one or more cells of the immune system is T cells.

8. The model of claim 7, wherein the first zone and the second zone are surrounded by a third zone, optionally wherein the third zone is a sinus.

9. The model of any one of claims 1-8, wherein the cell culture comprises one or more non-immune cell types, optionally stromal cells, endothelial cells, and/or neural cells, further optionally wherein the neural cells are neurons.

10. The model of any one of claims 1-9, wherein the cell culture comprises a scaffold, optionally wherein the scaffold is formed from a scaffold-forming composition that induces pattern formation, further optionally a photo-reactive crosslinking group and/or a photoinitiator; optionally wherein the scaffold-forming composition comprises a protein-based biomaterial scaffold-forming composition, further optionally a gelatin methacrylate, a gelatin norbornene, and/or a collagen; and/or optionally wherein the scaffold-forming composition comprises a non-protein material, further optionally polyethylene glycol.

11. The model of claim 10, wherein the cell culture comprises a mixture of scaffolds, optionally wherein the scaffolds are dispersed homogeneously through the cell culture or are patterned in one or more different zones of the cell culture.

12. The model of any one of claims 1-11, wherein the microfluidic housing comprises a material selected from the group consisting of a silicone rubber (optionally polydimethyl siloxane), glass, a thermoplastic polymer, a three-dimensional (3D) printed resin, a biomaterial (optionally a PEG-based material or a protein-based material), and a combination thereof.

13. The model of any one of claims 1-12, wherein the microfluidic housing comprises one or more surfaces having a surface chemistry modification.

14. The model of any one of claims 1-13, wherein the microfluidic housing comprises at least two channels in communication with the cell culture chamber, wherein at least one of the channels is configured to deliver input to the cell culture chamber and wherein at least one of the channels is configured to receive output from the cell culture chamber.

15. The model of any one of claims 1-14, wherein the microfluidic housing comprises one or more additional chambers in communication with the cell culture chamber, where one or more of the channels is configured to deliver input to the cell culture chamber from the one or more additional chambers and/or one or more of the channels is configured to deliver output from the cell culture chamber to the one or more additional chambers.

16. The model of any one of claims 1-15, wherein the one or more channels in communication with the cell culture chamber comprise one or more channels configured to deliver input to one or more zones in the cell culture, optionally wherein a first zone comprising a first type of the one or more cells of the immune system is surrounded by a second zone of a second type of the one or more cells of the immune system, further optionally wherein the first type of the one or more cells of the immune system is B cells and the second type of the one or more cells of the immune system is T cells.

17. The model of claim 16, wherein the first zone and the second zone are surrounded by a third zone, optionally wherein the third zone is a sinus.

18. The model of any one of claims 1-17, wherein the microfluidic housing comprises one or more additional layers comprising one or more additional channels and/or one or more additional chambers, wherein one or more additional layers are disposed above and/or below a first layer comprising the cell culture chamber and the one or more channels in communication with the cell culture chamber.

19. The model of claim 18, wherein the one or more additional channels and/or one or more additional chambers are in communication with the cell culture chamber and the one or more channels in communication with the cell culture chamber of the first layer, optionally wherein the communication is achieved via a valve, a port, and/or a semi-permeable membrane between the layers.

20. The model of any one of claims 1-19, comprising a length scale model of a mammalian lymph node, optionally wherein the lymph node is selected from the group consisting of a skin-draining lymph node, a cervical lymph node, a mesenteric lymph node, an iliac lymph node, a mediastinal lymph node, a popliteal lymph node, a tonsil, a Peyer's patch, and a tertiary lymphoid structure that forms in sites of chronic inflammation.

21. The model of any one of claims 1-19, comprising a model of spleen tissue.

22. The model of any one of claims 1-21, further comprising one or more pumps externally disposed relative to the microfluidic housing, the one more pumps communicating with the one or more channels for moving reagents into the one more channels.

23. A system comprising the model of any one of claims 1-22; and one or more other tissue-on-chip devices, wherein the model of any one of claims 1-21 is in communication with the one or more other tissue-on-chip devices, optionally wherein the system is used to model multi-tissue immunity and/or multi-tissue responses to a drug.

24. The system of claim 23, wherein the one or more other tissue-on-chip devices is selected from the group consisting of a liver device, a kidney device, a lung device, a brain device, and a tumor device.

25. A method for patterning cells in a culture on a microfluidic chip, the method comprising:
(a) providing a microfluidic housing comprising a cell culture chamber and one or more channels in communication with the cell culture chamber; and
(b) patterning cells in the cell culture chamber, optionally wherein the cells comprise one or more cells of the immune system, to provide a culture in the cell culture chamber.

26. The method of claim 25, wherein the culture is a three-dimensional (3D) culture.

27. The method of claim 25 or claim 26, wherein the patterning comprises directing a composition comprising one or more cells of the immune system to the cell culture chamber through the one or more channels; wherein the forming composition comprises a reagent that induces pattern formation, optionally wherein the composition comprises one or more non-immune cell types, optionally stromal cells, endothelial cells, neural cells such as but not limited to neurons, or any combination thereof.

28. The method of any one of claims 25-27, wherein the patterning comprises directing light through a photomask covering a portion of the microfluidic housing, providing one or more architectural features in the cell culture chamber, modifying surface chemistry of the microfluidic housing, and any combination thereof.

29. The method of claim 28, wherein the light directed through the photomask has a wavelength ranging from about 300 nanometers (nm) to about 800 nm, optionally about 405 nm.

30. The method of claim 28 or claim 29, wherein the photomask is selected from the group consisting of a printed transparency mask, a chrome mask, a digital mask, and another standard mask used for photolithography.

31. The method of claim 28, wherein the patterning comprises altering the surface chemistry of the microfluidic housing and wherein the reagent that induces pattern formation comprises a moiety that reacts with the altered surface chemistry.

32. A method for modeling an immune response of a subject, the method comprising:
(a) providing a model of a tissue of the immune system according to any one of claims 1-22 or a system according to claim 23 or claim 24;
(b) delivering a stimulus to the model; and
(c) evaluating a response to the stimulus.

33. The method of claim 32, wherein delivering the stimulus comprises delivering a vaccine to the model or system; and evaluating the response comprises evaluating one or more cell motility, gene expression, protein secretion, small molecule secretion, production of reactive oxygen species, and metabolic activity.

34. The method of claim 32, wherein delivering the stimulus comprises delivering an established or new therapy or drug to the model or system.

35. The method of claim 32, wherein delivering the stimulus comprises testing spatial organization of the model or system, optionally wherein delivering the stimulus comprises varying a location and/or an inclusion of a particular cell type between a first model or system and a subsequent model or system, and/or varying a rate and/or distribution of fluidic flow through the model or system.

36. The method of any one of claims 32-35, wherein the model is patterned using healthy and/or naive (resting) cells to model a healthy tissue of the immune system.

37. The method of any one of claims 32-35, wherein the model is patterned with cells from a diseased donor to model that disease.

38. The method of any one of claims 32-35, wherein the model is patterned with genetically modified cells to model a disease and/or to test a hypothesis about a role of a modified gene or pathway.
